(19) United States
(12) Patent Application Publication
(10) Pub. No.: US 2019/0231985 A1
(43) Pub. Date: Aug. 1, 2019

(54) Flush Syringe With Shielded Tip

(71) Applicant: Becton, Dickinson and Company, Franklin Lakes, NJ (US)

(72) Inventor: Roza Mahmoodian, New York, NY (US)

(21) Appl. No.: 16/253,739

(22) Filed: Jan. 22, 2019

Related U.S. Application Data
(60) Provisional application No. 62/622,458, filed on Jan. 26, 2018.

(51) Int. Cl. A61M 5/31

(52) U.S. Cl. CPC A61M 5/3137; A61M 2005/1403; A61M 2005/3104; A61M 5/3134

(57) ABSTRACT

Flush syringe assemblies are described herein. Such flush syringe assembly may include a barrel including a side wall defining a chamber, an open proximal end, a distal end having a distal wall with an elongate tip extending distally therefrom having a passageway therethrough in fluid communication with said chamber. A collar may be mounted on the distal wall of the barrel and surrounding the elongate tip. The flush syringe assembly may also include a removable cap.

### CROSS-REFERENCE TO RELATED APPLICATIONS

This application claims priority under 35 U.S.C. § 119(e) to U.S. Provisional Application No. 62/622,458, filed Jan. 26, 2018, the disclosures of which are incorporated herein by reference in their entirety.

### TECHICAL FIELD

The present disclosure generally relates to syringe assemblies, and particularly to syringe assemblies comprising a physical barrier to prevent contact of the syringe tip with the surrounding non-sterile environment. Embodiments of the present disclosure ensure adherence to aseptic techniques for use in flush procedures for vascular access devices (VAD's). Embodiments of the present disclosure are also directed to technology to reduce the risk for bloodstream infections (CRBSI) and intravenous (IV) line patency maintenance including capping technology, particularly for syringe assemblies for use in flush procedures.

### BACKGROUND

Vascular access devices (VADs) are commonly used therapeutic devices, which include peripheral catheters and central venous catheters. If not properly maintained or if exposed to a non-sterile environment, the VADs can become contaminated, sealed with blood clots or spread infection. To ensure VADs are used properly and do not become sealed or infected, protocols to ensure sterile practice have been developed. These protocols include sterilizing the VAD and flushing the catheter with a flush solution. Catheters are flushed using syringe assemblies filled with various fluids. In some cases, different fluids are injected sequentially in accordance with the protocol. For example, a saline solution followed by an anticoagulant such as heparin. The size of the syringe used to flush intravenous (I.V.) lines varies by various factors including the size and length of the catheter. Typically syringes of 1 ml, 3 ml, 5 ml and 10 ml volume are used. VAD protocols usually recommend flush procedures be performed after catheter placement, before fluid infusion, and before and after drug administration, blood sampling, transfusions and parenteral nutrition. The goal of these flush procedures is to confirm catheter patency, avoid drug incompatibilities, ensure the complete drug dose administration, prevent thrombus formation and minimize the risk of blood stream infections.

Conventional flush syringes have a barrel with a luer tip at one end which is exposed to the non-sterile environment once the syringe tip is removed from packaging thus providing an opportunity for undesired contamination. Consequently, there is a need for a syringe, particularly a flush syringe, providing a physical barrier around the syringe tip which promotes aseptic practice by reducing or eliminating “touch” contamination of the syringe, particularly the tip of a syringe, with the surrounding non-sterile environment.

### SUMMARY

One aspect of the present disclosure pertains to a flush syringe assembly including a barrel having a side wall with an inside surface defining a chamber for retaining a fluid, an open proximal end, a distal end including a distal wall with an elongate tip extending distally therefrom having a passageway therethrough in fluid communication with said chamber. The flush syringe assembly also includes a collar mounted on the distal wall of the barrel and surrounding the elongate tip. The collar may include at least one side wall having an inside surface defining a compartment, an open distal end, a proximal end adjacent the distal wall of the barrel. The flush syringe assembly may also include an elongated plunger rod disposed within the barrel, the plunger rod comprising a distal portion and a proximal portion. The plunger rod may also include a distal end including a stopper slidably positioned in fluid-tight engagement with the inside surface of the barrel for drawing fluid into and driving fluid out of the chamber by movement of the stopper relative to the barrel. The elongated plunger rod may extend outwardly from the open proximal end of the barrel.

In one or more embodiments, the distal wall of the barrel includes a plurality of threads to connect the barrel to the collar. In certain embodiments, the proximal end of the collar may be threaded onto the distal wall of the barrel.

In one or more embodiments, the compartment of the collar surrounds the elongated tip. In one or more embodiments, the collar may be removable.

In one or more embodiments, the collar may have a convex inner surface or a concave inner surface.

In one or more embodiments, the collar has a shape of a trapezoidal prism.

In one or more embodiments, the fluid is a flush fluid.

One aspect of the present disclosure pertains to a flush syringe assembly including a barrel having a side wall with an inside surface defining a chamber for retaining a fluid, an open proximal end, a distal end including a distal wall with an elongate tip extending distally therefrom having a passageway therethrough in fluid communication with said chamber. A collar may be mounted on the distal wall of the barrel and surround the elongate tip. The collar may include at least one side wall having an inside surface defining a compartment, an open distal end, a proximal end adjacent the distal wall of the barrel. A removable cap having a body, a proximal end, and a closed distal end may be mounted on the distal end of the collar. An elongated plunger rod may be disposed within the barrel, the plunger rod comprising a distal end and a proximal end. A stopper may be slidably disposed on the distal end of the plunger rod and positioned in fluid-tight engagement with the inside surface of the barrel for drawing fluid into and driving fluid out of the chamber by movement of the stopper relative to the barrel.

In one or more embodiments, the compartment of the collar surrounds the elongated tip.

In one or more embodiments, the distal wall of the barrel may include a plurality of threads to connect the barrel to the collar. In one or more embodiments, the distal wall of the collar may include a plurality of threads to connect the collar to the removable cap.

In one or more embodiments, the collar may be removable.

In one or more embodiments, the collar may have a convex or concave inner surface. In a specific embodiment, the collar has a shape of a trapezoidal prism.

In one or more embodiments, the fluid is a flush fluid

In one or more embodiments, the removable cap may include an outward protrusion extending from the body of the cap and corresponding with the opening of the distal end of the elongate tip.

In one or more embodiments, the removable cap has a cross-sectional shape that is triangular, square, pentagonal, hexagonal, heptagonal, octagonal, symmetric or non-symmetric polygonal.

### BRIEF DESCRIPTION OF THE DRAWINGS

FIG. 1 illustrates a partial perspective view of a flush syringe with a collar in accordance with one or more embodiments of the present disclosure;

FIG. 2 illustrates a top perspective view of a flush syringe with a collar in accordance with one or more embodiments of the present disclosure;

FIG. 3 illustrates a side view of a flush syringe with a collar and cap in accordance with one or more embodiments of the present disclosure;

FIG. 4 illustrates a cross-sectional perspective view of a flush syringe with a collar and a cap in accordance with one or more embodiments of the present disclosure;

FIG. 5 illustrates a top perspective view of a flush syringe with a collar in accordance with one or more embodiments of the present disclosure;

FIG. 6 illustrates a side view of a flush syringe with a collar and cap in accordance with one or more embodiments of the present disclosure;

FIG. 7 illustrates a cross-sectional perspective view of a flush syringe with a collar and a cap in accordance with one or more embodiments of the present disclosure;

FIG. 8 illustrates a perspective view of an assembled flush syringe with a collar and a cap in accordance with one or more embodiments of the present disclosure; and

FIG. 9 illustrates a perspective view of a flush syringe with a collar and a cap that is removed in accordance with one or more embodiments of the present disclosure.

### DETAILED DESCRIPTION

Before describing several exemplary embodiments of the present disclosure, it is to be understood that the disclosure is not limited to the details of construction or process steps set forth in the following description. The disclosure is capable of other embodiments and of being practiced or being carried out in various ways.

With respect to terms used in this disclosure, the following definitions are provided.

Reference to “flush syringe assembly” includes syringes that are indicated for use in the flushing of VADs. The practice of flushing ensures and maintains catheter patency and helps prevent the mixing of incompatible pharmaceuticals.

As used herein, the use of “a,” “an,” and “the” includes the singular and plural.

As used herein, the term “catheter related bloodstream infection” or “CRBSI” refers to any infection resulting from the presence of a catheter or IV line.

As used herein, the term “Luer connector” refers to a connection collar that is the standard way of attaching syringes, catheters, hubbed needles, IV tubes, etc. to each other. The Luer connector consists of male and female interlocking tubes, slightly tapered to hold together better with even just a simple pressure/twist fit. Luer connectors can optionally include an additional outer rim of threading, allowing them to be more secure. The Luer connector male end is generally associated with a flush syringe and can interlock and connect to the female end located on the VAD. A Luer connector comprises a distal end, a proximal end, an irregularly shaped outer wall, a profiled center passageway for fluid communication from the chamber of the barrel of a syringe to the hub of a VAD. A Luer connector also has a distal end channel that releasably attaches the Luer connector to the hub of a VAD, and a proximal end channel that releasably attaches the Luer connector to the barrel of a syringe.

Because clinicians have to handle multiple components while flushing intravenous (IV) catheters, a certain level of dexterity is required to carry out the procedure while preventing the syringe tip from coming into contact with the surrounding environment. If the syringe tip touches any non-sterile surfaces, “touch” contamination can occur which can cause microbial growth in the IV line and consequently lead to incidents of catheter-associated-bloodstream infection (“CRBSI”) and central line—associated bloodstream infection (“CLABSI”) which are very costly and lethal. Embodiments of the present disclosure relate to a flush syringe having a collar surrounding the elongate tip of the syringe. The collar provides a physical barrier around the syringe tip. Because of its shape, the collar of the present disclosure can help facilitate the alignment of the flush syringe with the catheter hub and/or needle-free connector and thus reduce the chances of “touch” contamination.

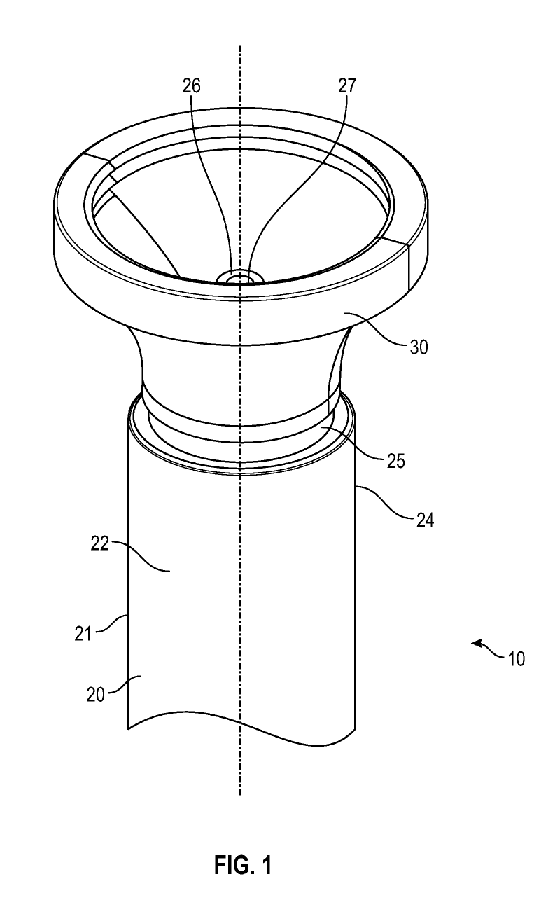

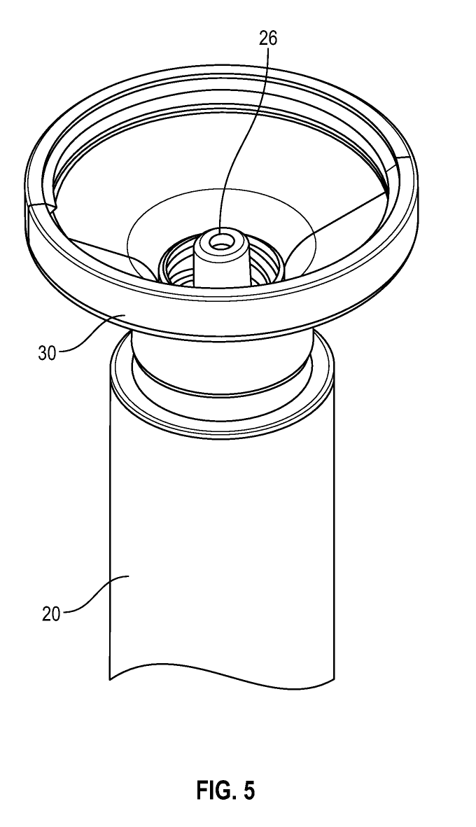

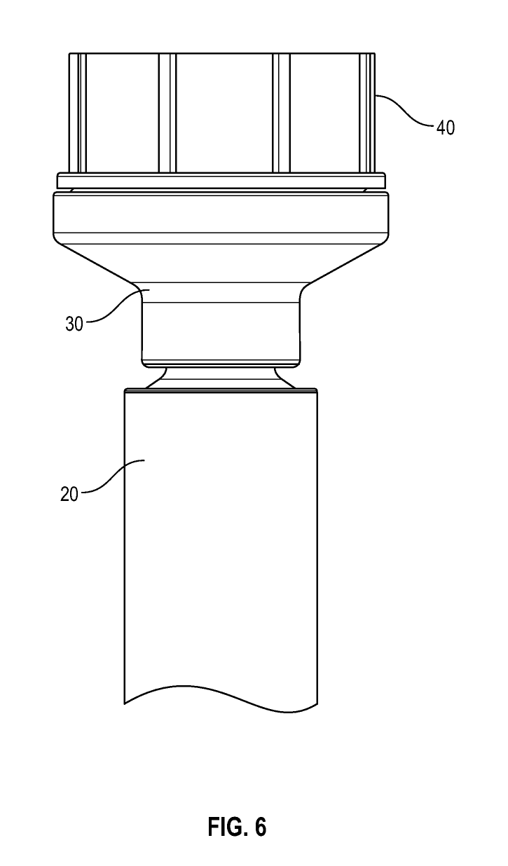

Provided are syringe assemblies that include a plunger rod and a syringe barrel having an open proximal end and a distal tip, the distal syringe tip surrounded by a collar to facilitate alignment of the syringe with a catheter hub or needle-free connector, as well as, reducing contamination of the syringe by preventing contact of the syringe tip with the surrounding non-sterile environment. Referring to FIGS. 1-7, a syringe assembly 10 according to the present disclosure generally comprises a barrel 20, including a side wall 21 having an inside surface defining a chamber 22 for retaining a fluid. In one or more embodiments, the fluid is a flush fluid. The barrel 20 further includes an open proximal end (not shown) and a distal end 24 having a distal wall 25 with an elongated tip 26 extending distally therefrom and having a passageway 27 therethrough in fluid communication with the chamber, the distal wall adapted for connection to a collar 30. The open proximal end of the barrel is disposed on the opposite end of the distal end 24. The distal wall 25 may comprise a plurality of threads 28 for attachment to the collar 30, however the collar 30 may engage onto the distal wall via a variety of methods, including press-fitting, adhering, detenting, co-injection molding, etc.

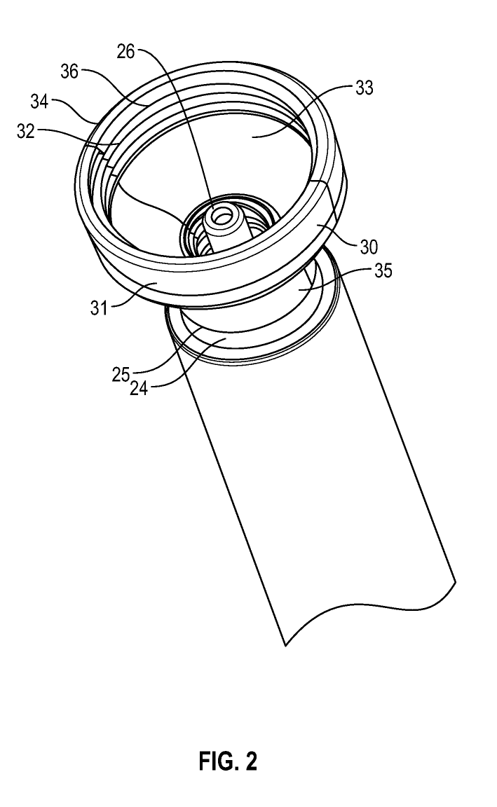

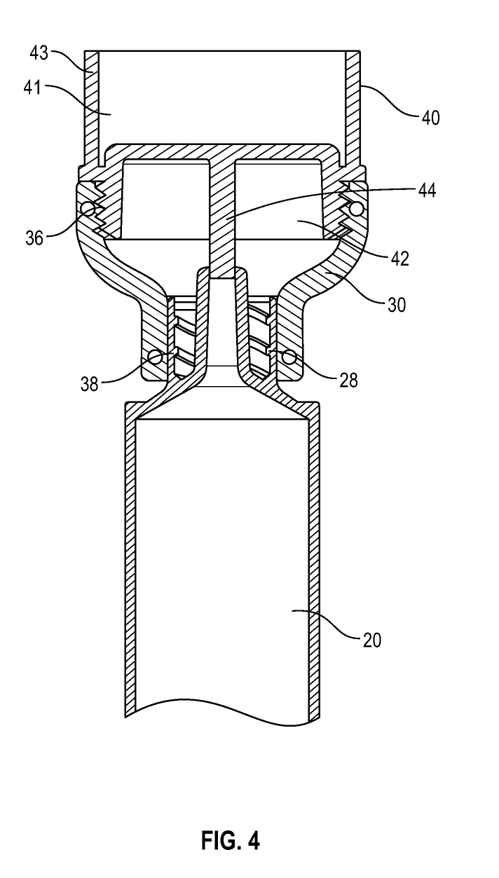

As shown in FIG. 2, collar 30 mounted on the distal end 24 of the barrel, the collar 30 including at least one side wall 31 having an inside surface 32 defining a compartment 33 surrounding the elongated tip 26, a open distal end 34, and a proximal end 35 adjacent the distal wall 25 of the barrel. Elongated tip 26 adapted for connection to a hub of a vascular access device. The collar 30 may comprise a plurality of threads 36 on the inside surface for connection to a vascular access device. As shown in FIGS. 4, 5 and 7, the collar 30 may comprise an engagement portion 38 on the proximal end 35 for attachment to the distal wall 25 of the barrel. Upon manufacture, the flush syringe assembly 10 can be provided with the collar 30 connected at the proximal end 35 onto the distal wall 25 of the barrel. The engagement may be via a variety of methods not limited to: threading, press-fitting, adhering, detenting, co-injection molding, etc. The shape of the collar 30 can vary. Collar 30 may have shapes including, but not limited to, a convex inner surface (for example a paraboloid), concave inner surface, with a straight profile (i.e., semi conical shape), or have the shape of a trapezoidal prism. The length of this extension from the main body of syringe and the degree of openness/straightness of the profile (how wide the collar is at the end farthest from the syringe barrel) can vary.

In one or more embodiments, the collar 30 may be removable from the syringe assembly 10.

In an embodiment, the collar 30 may comprise a plurality of threads adapted for connection to the hub of the vascular access device. In one or more embodiments, the collar 30 surrounds an elongate tip adapted for connection to the hub of the vascular access devices. In one or more embodiments, the elongate tip is a Luer tip.

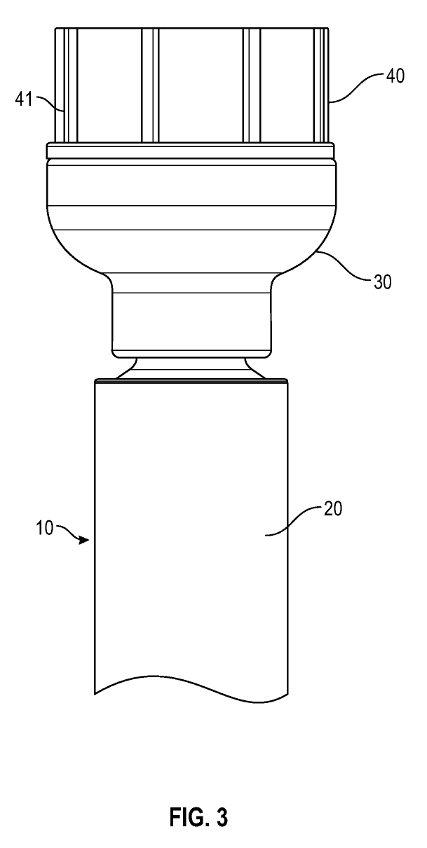

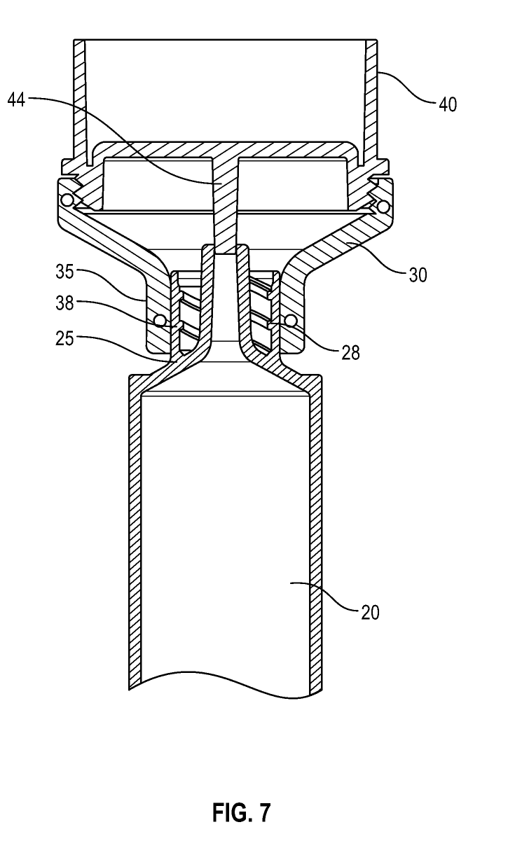

As shown in FIGS. 3, 4 and 6, the open distal end 34 of the collar 30 may comprise a removable cap 40. The removable cap 40 includes a body 41 with a proximal end 42, a closed distal end 43. The distal end 43 and the proximal end 42 define the length of the cap 40. FIGS. 3, 4, 6 and 7 show a side view and a cross-sectional view, respectively, of a cap 40 in accordance with one or more embodiments of the present disclosure. It should be noted that cap distal wall 430 may be recessed within the cap distal end 43, such that a cap recess 431 at cap distal end 43 is formed. Upon assembly, the proximal end 42 of the cap 40 is adjacent the distal end 34 of the collar 30. As shown in FIGS. 4 and 7, cap 40 may comprises an outward protrusion 44 that extends from the body 41 of the cap 40 and corresponds with the opening of the distal end of the elongate tip 26. The proximal end 42 of removable cap 40 may comprise a plurality of threads on the outside surface of the cap for attachment to corresponding grooves disposed on the inside surface of the distal end of the collar 30. In one or more embodiments, at least one screw thread adapted to allow the cap to be screwed onto collar 30.

The cross-sectional shape of the cap 40 can be any suitable shape including, but not limited to, triangular, square, pentagonal, hexagonal, heptagonal, octagonal, symmetric or non-symmetric polygonal. The shape of the cap 40 can provide a comfortable feel for the user and enhanced gripping ability to allow the user to easily connect or disconnect the cap from the collar 30. In some embodiments, the cap 40 is irregularly shaped. As used in this specification and the appended claims, the term “irregularly shaped” means that the cross-sectional shape provides a surface or edge that is detrimental to free rotation about the cross-section. For example, a hexagon or oval shape would be considered “irregular”.

The syringe assembly 10 may be filled with flush solution using known methods. Additionally, the syringe assembly 10 may be provided pre-filled from the manufacturer or supplier. The flush solution may be any solution intended for flushing or maintaining performance of VAD's. It is preferred that the flush solution be selected from the group consisting of saline flush solution and heparin lock flush solution. These solutions are known in the art and are readily available. An example of a saline flush solution includes, but is not limited to, 0.9% sodium chloride USP for injection. An example of a heparin lock flush solution includes but is not limited to 0.9% sodium chloride with 100 USP units of heparin sodium per mL or 10 USP units of heparin sodium per mL.

Once the connection of the syringe assembly 10 to the VAD is completed, fluid communication from the barrel 20 of the syringe to the vascular access device can occur. Fluid is drawn from the barrel 20 through the integral passageway 27 into the IV or catheter. Because of the presence of the collar 30, fluid communication through a vascular access device and into a patient is conducted under aseptic conditions without any additional swabbing steps and diligence on the part of the clinician.

In one or more embodiments, the collar 30 can be integrally formed on the distal wall 25 of the syringe barrel 20 for fluid communication to the vascular access device.

The barrel may also include a tip which extends distally from the barrel. The tip can have an outer diameter that is different from or the same as the outer diameter of the rest of the barrel. For example, as shown in the Figures, the outer diameter of the tip has a smaller outer diameter than the barrel portion that is proximal of the tip. The tip of the barrel may include a luer slip connection or a locking luer type collar concentrically surrounding the tip or within the tip.

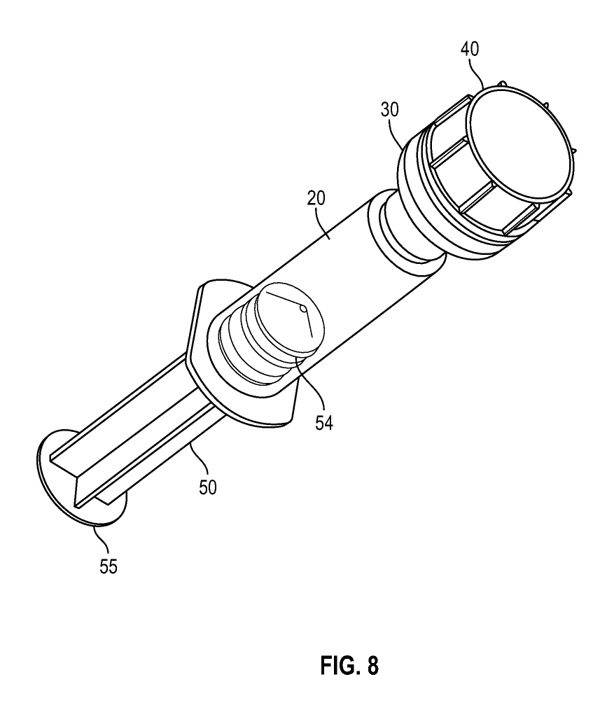

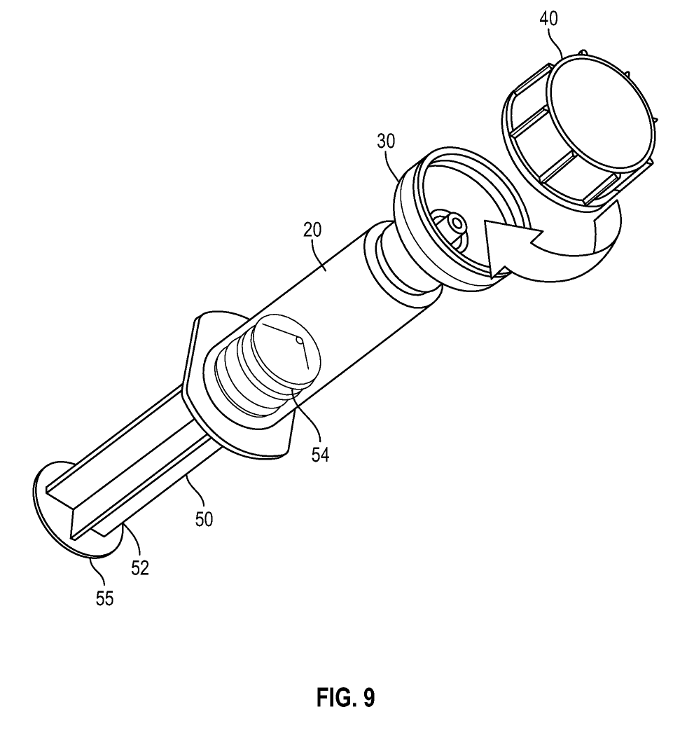

FIG. 8 illustrates a perspective view of an assembled flush syringe with a collar and a cap in accordance with one or more embodiments of the present disclosure. FIG. 9 illustrates a perspective view of a flush syringe with a collar and a cap that is removed in accordance with one or more embodiments of the present disclosure.

An elongated plunger rod 50 may include a distal portion and a proximal portion 52, the plunger rod further comprising a distal end 51 including a stopper 54 slidably positioned in fluid-tight engagement with the inside surface of the barrel for drawing fluid into and driving fluid out of the chamber by movement of the stopper relative to the barrel, the elongated plunger rod extending outwardly from the open proximal end 23 of the barrel, the stopper having a distal surface.

An elongate plunger rod may be disposed within the barrel 20. The plunger rod includes an elongate body portion with a proximal end and a distal end.

The elongate body portion of the plunger rod has an axial length extending from the proximal end to the distal end. The body portion may include a single beam or features, which may have cylindrical or other shapes. The body portion may be formed by two perpendicularly intersecting beams.

The plunger rod 50 may also include a thumb press 55 at the proximal end 52 of the elongate body portion. The shape of the thumbpress can vary depending on the desired usage of the flush syringe assembly. The shape of the thumb press may be round, square, rectangular, triangular, oval, pentagonal, hexagonal and cruciform.

A stopper 54 can be connected to the distal end of the plunger rod. The shape and size of the stopper can be any suitable shape or size depending on, for example, the shape and size of the barrel and plunger rod. The plunger rod 50 is slidably positioned in the barrel 20 so that the stopper is in fluid-tight contact with the inside surface of the barrel and so that distal movement of the plunger rod 50 relative to the barrel 20 causes the stopper 54 to push the fluid out of the barrel. In some embodiments, the stopper is slidably positioned in fluid-tight contact with the inside surface of the barrel for driving fluid out of the chamber by movement of the stopper relative to the barrel. The stopper can be connected to the distal end of the elongate plunger rod by any suitable means. In some embodiments, the stopper is connected by a mechanical connection such as interaction of complementary screw threads and press-fit connections. The stopper may be slidably positioned in fluid-tight engagement with the inside surface of the barrel for drawing fluid into and driving fluid out of the chamber.

The stopper may be made of any material suitable for providing a seal with the inside surface of the barrel. For example, the stopper may be made of thermoplastic elastomers, natural rubber, synthetic rubber or thermoplastic materials and combinations thereof. The stopper may be integrally formed or composed of separate components of the same or different materials joined together. The plunger rod may be made of material which is more rigid than the stopper such as polypropylene, polyethylene and the like. Materials should be chosen to be compatible with the procedure being used.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as disclosed.

What is claimed is:

1. A flush syringe assembly comprising:
a barrel including a side wall having an inside surface defining a chamber for retaining a fluid, an open proximal end, a distal end including a distal wall with an elongate tip extending distally therefrom having a passageway therethrough in fluid communication with said chamber;
a collar mounted on the distal wall of the barrel and surrounding the elongate tip, the collar including at least one side wall having an inside surface defining a compartment, an open distal end, a proximal end adjacent the distal wall of the barrel; and
an elongated plunger rod disposed within the barrel, the plunger rod comprising a distal end and a proximal end, the distal end including a stopper slidably positioned in fluid-tight engagement with the inside surface of the barrel for drawing fluid into and driving fluid out of the chamber by movement of the stopper relative to the barrel, the elongated plunger rod extending outwardly from the open proximal end of the barrel, the stopper having a distal surface.

2. The flush syringe assembly of claim 1, wherein the distal wall of the barrel includes an engagement portion to connect the barrel to the collar.

3. The flush syringe assembly of claim 2, wherein the proximal end of the collar is threaded onto the distal wall of the barrel.

4. The flush syringe assembly of claim 1, wherein the compartment of the collar surrounds the elongated tip.

5. The flush syringe assembly of claim 1, wherein the collar is removable.

6. The flush syringe assembly of claim 1, wherein the collar has a convex inner surface.

7. The flush syringe assembly of claim 1, wherein the collar has a concave inner surface.

8. The flush syringe assembly of claim 1, wherein the collar has a shape of a trapezoidal prism.

9. The flush syringe assembly of claim 1, wherein the fluid is a flush fluid.

10. A flush syringe assembly comprising:
a barrel including a side wall having an inside surface defining a chamber for retaining a fluid, an open proximal end, a distal end including a distal wall with an elongate tip extending distally therefrom having a passageway therethrough in fluid communication with said chamber;
a collar mounted on the distal wall of the barrel and surrounding the elongate tip, the collar including at least one side wall having an inside surface defining a compartment, an open distal end, a proximal end adjacent the distal wall of the barrel;
a removable cap having a body, a proximal end, and a closed distal end, the removable cap being mounted on the distal end of the collar;
an elongated plunger rod disposed within the barrel, the plunger rod comprising a distal end and a proximal end; and
a stopper slidably disposed on the distal end of the plunger rod and positioned in fluid-tight engagement with the inside surface of the barrel for drawing fluid into and driving fluid out of the chamber by movement of the stopper relative to the barrel.

11. The flush syringe assembly of claim 10, wherein the compartment of the collar surrounds the elongated tip.

12. The flush syringe assembly of claim 10, wherein the distal wall of the barrel includes a plurality of threads to connect the barrel to the collar.

13. The flush syringe assembly of claim 10, wherein the distal wall of the collar includes a plurality of threads to connect the collar to the removable cap.

14. The flush syringe assembly of claim 10, wherein the collar is removable.

15. The flush syringe assembly of claim 10, wherein the collar has a convex inner surface.

16. The flush syringe assembly of claim 10, wherein the collar has a concave inner surface.

17. The flush syringe assembly of claim 10, wherein the collar has a shape of a trapezoidal prism.

18. The flush syringe assembly of claim 10, wherein the fluid is a flush fluid.

19. The flush syringe assembly of claim 10, wherein the removable cap includes an outward protrusion extending from the body of the removable cap and corresponding with the passageway on the distal end of the elongated tip.

20. The flush syringe assembly of claim 10, wherein the removable cap has a cross-sectional shape that is triangular, square, pentagonal, hexagonal, heptagonal, octagonal, symmetric or non-symmetric polygonal.
